(19) United States
(12) Patent Application Publication
(10) Pub. No.: US 2018/0207326 A1
(43) Pub. Date: Jul. 26, 2018

(54) Coated endovascular devices

(71) Applicant: Daniel E. Walzman, Bergenfield, NJ (US)

(72) Inventor: Daniel E. Walzman, Bergenfield, NJ (US)

(21) Appl. No.: 15/932,592

(22) Filed: Mar. 20, 2018

Related U.S. Application Data
(63) Continuation-in-part of application No. 15/732,365, filed on Oct. 30, 2017.
(60) Provisional application No. 62/497,851, filed on Dec. 5, 2016.

(51) Int. Cl. A61L 31/14; A61L 31/08; A61L 31/16; A61L 27/52; A61L 27/54; A61L 27/58; A61L 27/28

(52) U.S. Cl. CPC A61L 31/145; A61L 31/08; A61L 31/148; A61L 31/16; A61L 27/52; A61L 2300/606; A61L 27/58; A61L 27/28; A61L 2300/40; A61L 2300/416; A61L 2300/604; A61L 27/54

(57) ABSTRACT

The present disclosure relates to the field of medical treatment. More particularly, the current invention delivers medication via a coating via a hydrogel infused with pharmaceutical compounds into the bloodstream to promote the supply to the distal vascular bed beyond the stent. The present invention uses in a preferred embodiment a stent with a thin coating of biodegradable or non-biodegradable hydrogel designed to ameliorate or eliminate vasospasms or thromboses, or to treat cancer, which hydrogel may optionally be impregnated with pharmaceutical compounds. The present invention also teaches the use of thin hydrogel coatings to ameliorate treatment related difficulties.

### CROSS-REFERENCES

This is a continuation-in-part application claiming priority to non-provisional patent application Ser. No. 15/732,365 filed Oct. 30, 2017 for an “Alternate use for Hydrogel Intrasaccular Occlusion Device”, and to provisional patent application Ser. No. 62/497,851 filed Dec. 5, 2016 for a “Hydrogel Intrasaccular Occlusion Device” (Walzman).

### FEDERALLY FUNDED R&D

None

### BACKGROUND OF THE INVENTION

### Field of Invention

The present disclosure relates to the field of stent treatment. More particularly, the present invention is an apparatus directed to preventing reducing blood and tissue reactions to a foreign body, as well as a means of targeted drug delivery into a target vascular territory for the treatment of various diseases such as cancer and vasospasms deploying coated devices such as stents.

### Background Art

Vasospasm is a common complication that follows aneurysmal subarachnoid hemorrhage (SAH). Prior art comprehensively describes the deficits accompanying vasospasm and, most importantly, made the association between vasospasm and neurological deficits, also known as delayed ischemic deficits (DID).

Vasospasm is one of the leading causes of morbidity and mortality following aneurysmal subarachnoid hemorrhage (SAH). Radiographic vasospasm usually develops between 5 and 15 days after the initial hemorrhage, and is associated with clinically apparent delayed ischemic neurological deficits (DID) in one-third of patients.

The pathophysiology of this reversible vasculopathy is not fully understood but appears to involve structural changes and biochemical alterations at the levels of the vascular endothelium and smooth muscle cells. One theory is that blood in the subarachnoid space trigger these changes. In addition, cerebral perfusion may be concurrently impaired by hypovolemia and impaired cerebral auto-regulatory function.

Another cause of cerebral vasospasm is a spontaneous or secondary reversible vasoconstriction syndrome not associated with an acute subarachnoid hemorrhage.

The combined effects of these processes can lead to reduction in cerebral blood flow so severe as to cause ischemia leading to infarction. One therapy involves the use of oral Nimodipine, a calcium channel antagonist. It is known to reduce the impact of DID.

Additionally therapy combining hemodynamic augmentation, transluminal balloon angioplasty, and intra-arterial infusion of vasodilator drugs is also know to reduce the impact of DID. However, these drugs delivered via directed catheter endovascular therapy, are short lasting, which often requires multiple procedures, and limits the overall efficacy. The prior art discloses several drugs, with different mechanisms of action, also may ameliorated the impact of DID.

A drug-eluting stent (DES) is a well known medical device. The prior art discloses that a DES is a peripheral or coronary stent placed into narrowed, diseased peripheral or coronary arteries that slowly releases a drug to block cell proliferation. Said release may prevent fibrosis that, together with clots (thrombi), could otherwise block the stented artery. The DES device is usually placed within the peripheral or coronary artery.

Drug-eluting stents in current clinical use were approved by the FDA after clinical trials showed they were statistically superior to bare-metal stents for the treatment of native coronary artery narrowings, having lower rates of major adverse cardiac events such as myocardial infarctions. The first drug-eluting stents to be approved in the U.S. were coated with Paclitaxel or an mTOR inhibitor, such as Sirolimus.

The use of DES decreases the risk of in-stent stenosis by preventing fibrosis. The use of DES, however, also delays endothelial regrowth over the stent, and is associated with an increased risk of in-stent thrombosis.

The use of covering the surface of a stent with hydrogel and/or the use of hydrogel infused with certain medications may ameliorate or eliminate the tendency of stents to thrombose without the use of dual antiplatelet therapy, as well as the tendency of stents to cause a tissue reaction at the implantation site that can lead to in-stent stenosis, whether in a vessel, ureter, bile duct, or other organ. The use of a thin layer of hydrogel covering the surfaces of stents can reduce or eliminate the thrombogenic tendency, thus reducing or eliminating the need for antiplatelet therapy and their associated risk, as well as reducing or eliminating undesired thrombus formation.

Additionally medications can be adhered directly to stents and/or in hydrogel lining stents, so that a prescribed dose of said medication will be released over a prescribed time, for a local effect in that particular vessels perfusion territory. This local selective delivery will often reduce undesired systemic effects of said medication, while often increasing local efficacy.

Stents and other endovascular devices such as mechanical cardiac valves can have consequences in that they are thrombogenic when first inserted, until they are incorporated into the vessel/endothelialized, or in some cases permanently. This results in significant rates of thrombotic complications, including thrombosed vessels resulting in stroke, myocardial infarction, or other ischemic complications. In order to minimize such risks patients are routinely started on antiplatelet therapy, often dual antiplatelet therapy with agents such as Plavix (clopidogrel) or BRILINTA® (ticagrelor), and aspirin. Alternatively, as is the case with many cardiac valves, patients are started on anticoagulants, such as Coumadin.

In addition, other endovascular devices, particularly those implanted in the heart such as mechanical heart valves, tend to cause a different type of clot that necessitates the use of anticoagulants to protect against clot formation. Although the medications reduce the rate of clot formation, they do not eliminate clot formation altogether and patients can still suffer complications from clotting. Still further, all these medications have significant rates of bleeding complications.

Hydrogel is more inert than metals and some plastics and other common materials, and does not cause thrombus formation/induction. Furthermore, a study published in the Journal of the American College of Cardiology: Basic to Translational Science, reported that an injectable gel can maintain its healing characteristics. In particular, rebuilding of muscular structures was reported from a gel originally derived from a pig's cardiac muscle tissue, which was stripped of cells until all that was left was an extracellular matrix. A 2010 study in the Journal of Cell Science noted that an element of gel used in the aforementioned Journal of the American College of Cardiology study was responsible for tissue regeneration and re-growth: One non-limiting version of a hydrogel that expands in the body is a co-polymer of acrylamide and sodium acrylate cross linked.

The prior art teaches uniform distribution of medication along the stent. For example, U.S. Pat. No. 8,367,151 (O'Brien et al.) teaches a sputtered coating “that is evenly distributed over the outward-facing side of the stent's wire mesh” (Abstract). Such even distribution is ideal for treating tissue in proximity to the stent. It is suboptimal for the treatment of tissue downstream of the stent.

It is, therefore, desirable to have devices incorporating a hydrogel coating, usually a thin coating of same in interventional, intravascular and other luminal organ therapies.

There is a need for a broader spectrum of stents with an adhered “medium” that carries and slowly releases medication into blood stream (as opposed to current medicated stents, whose medication is designed to inhibit local tissue reaction to reduce intimal hyperplasia and in-stent stenosis). There is a need to release medication into blood, over a prescribed time, rather than using such medication for a purely local effect.

The haemodynamics of arterial blood flow describes the mechanical properties and the interactions between blood flow and vascular walls. More specifically, haemodynamics describe the kinematics and dynamics of blood flow and obey the laws that govern the mass and momentum of fluid elements.

The blood vessels have the ability to respond to haemodynamic stimuli, including the stretch and shear stress resulting from flow and circulatory (transmural) pressure. This ability is mainly mediated by vascular endothelial cells, which are in direct contact with blood flow.

In fluid dynamics, laminar flow (or streamline flow) occurs when a fluid flows in parallel layers, with no disruption between the layers. At low velocities, the fluid tends to flow without lateral mixing, and adjacent layers slide past one another like playing cards. There are no cross-currents perpendicular to the direction of flow, nor eddies or swirls of fluids. In laminar flow, the motion of the particles of the fluid is very orderly with particles close to a solid surface moving in straight lines parallel to that surface.

When a fluid is flowing through a closed channel such as a blood vessel either of two types of flow may occur depending on the velocity and viscosity of the fluid: laminar flow or turbulent flow. Laminar flow tends to occur at lower velocities, near the wall of a closed channel. Turbulent flow tends to occur at the end of a tube.

Turbulent flow is a less orderly flow regime that is characterized by eddies or small packets of fluid particles, which result in lateral mixing. In some embodiments, the present invention uses this physical phenomenon to move small packets of pharmacological compounds from the outer surface of the present invention to the center of a vessel channel and thus further downstream than eluting stents disclosed by the prior art.

The prior art discloses stents which release medications. However, said stents are specifically designed to deliver pharmaceutical compounds to the local area, immediately at the sight of stent implantation, wherein the medication is released.

Said prior art discloses stent-eluding medication slowly released directly into the adhered tissue of the vessel wall, rather than into the blood. Any minute amount of medication flowing into the blood and downstream is an unintended consequence. The prior art indicates that said downstream waste should be minimized by the use of fast-acting medications.

The prior art discloses structures which enhance release of medication to tissue proximal to the stent, whereas the present invention is designed to minimize medication proximal to the stent. In some embodiments present invention does so by infusing a higher density of medication at the downstream end than the upstream end of the stent, such that blood is channeled around the downstream concentration. The stent is designed to cause turbulent flow just beyond the downstream edge of the device, causing the medication to flow from the outer surface of the downstream end of the device into the center of the vessel. As a result, the outer coating is channeled to the turbulent flow (which is in the center of the vessel) rather than the laminar flow stream, thus enhancing the distance the medication can flow before it settles on the vessel wall or bed.

In other embodiments the preponderance of the medication is on the internal surface of the stent, in direct contact with the blood as it continuously flows through the stent, rather than on the outer surface of the stent. This facilitates release of the drug into the bloodstream.

The present invention substantially addresses the foregoing unmet needs.

### SUMMARY OF THE INVENTION

The current invention places a thin coating of hydrogel on the entire surface of any endovascular device exposed to the inner surface of the blood vessel and/or blood products. This should preferably include placing a thin layer of hydrogel over the surfaces exposed to tissue as well. The former may reduce the risk of thrombus formation, and the latter may reduce the risk of unwanted tissue reactions and/or in-stent stenosis.

In other non-vascular embodiments, the hydrogel can be on all layers, or on the outer surface alone. When the inner surface of the stent is not exposed to blood, there is sometimes less need to prevent interaction of the inner surfaces of the stent.

By completely covering these devices with the thin layer of hydrogel, medical practitioners can significantly reduce the rate of thrombus formation and thus reduce the need for antiplatelet and or anticoagulant. Antiplatelet and anticoagulant medications have significant associated morbidity as well. By eliminating the need for them morbidity can be reduces further. Furthermore, by reducing exposure of the stent or other medical device to the tissue in which it is implanted, local tissue reactions, which can cause in-stent stenosis, scar tissue, and other complications, can often be avoided.

A thin layer of hydrogel placed on any vascular stent (e.g., cardiac or other) reduces a tendency for thrombosis, and further reduces the need for dual antiplatelet therapy. Reducing interaction between metal and tissue in both vascular and non-vascular stents also reduces rates of intimal hyperplasia and in-stent stenosis.

Non limiting examples of endovascular devices that can be covered with such a layer of hydrogel include metal stents, covered stents, cardiac valves, left atrial appendage occlusion devices such as the Watchman, intra-saccular aneurysm devices (such as U.S. application Ser. No. 15/732,365), pressure monitors, wires, and leads. The prior art discloses that medical devices with exposed metal is inherently dangerous, and further discloses covering metals with plastics, and/or polyesters, and/or Dacron, among other coverings. Prior art discloses hydrogel for plugging aneurysms but has not disclosed hydrogel as an agent for covering metal because most forms of hydrogel do not readily adhere to metal. The present invention employs hydrogel for the purpose of coating metal, plastics, and other implantable foreign materials. The present invention also deploys hydrogel adhered to implantable medical devices as well as for delivering medication as well.

If hydrogel is placed around all surfaces, including the surface pressing on the vessel or other organ wall, it will help reduce the rate of intimal hyperplasia caused by the tissue reacting to the foreign body. Intimal hyperplasia causes luminal narrowing and/or occlusions. In some cases, pressure may appear on the outer wall as well.

The present invention in all embodiments will be partially or fully covered by a thin coating of hydrogel. Said coating can act as a barrier to reduce the thrombogenicity of the stent when places intravascular, reducing thrombotic complications, and reducing the need for antiplatelets (with their associated potential hemorrhagic complications—especially in patients with a recent incident of brain-bleeding). Said coating can also reduce tissue reaction to the stents. Additionally, said coating can act as a medication delivery system.

The present invention teaches the affixation of at least one hydrogel layer that in vivo to any or all surfaces of a stent made of at least one material adapted from a stent and other medical devices for various uses in the human or animal body. For purposes of this invention, hydrogel coating will be in a nonhydrated state in packaging, and will be hydrated to its full expansion either immediately before implantation, or it will preferably expand in the bodyuse, upon exposure to bodily fluid.

The current invention may be used in the brain, the peripheral vasculature, and the cardiac vasculature. It may be used in arteries, veins, and lymph vessels. It may be used in ureters, tracheas, bile ducts, and other luminal organs.

The present invention discloses a device for any stent to which any medication is directly adhered or adhered to a medium that is adhered to the stent, wherein said medication releases into the blood (i.e. the medication is delivered via the stent into the blood and to the vascular bed supplied by that vessel, and to the tissue of that vascular bed), in contrast to current drug-eluting stents, wherein the drug is on the stent for the local tissue affect of the drug, not for release into the blood and downstream therapy. One example of a medium to carry said medication for release is hydrogel.

Cardiac prosthesis is any material surgically implanted in the heart to replace a heart element that has become damaged due to heart valve disease. Cardiac prosthesis include but are not limited to valves, wires, pumps, and stents. Said cardiac prosthesis may also be coated with hydrogel.

### BRIEF DESCRIPTION OF THE DRAWINGS

The invention will be better understood and objects other than those set forth above will become apparent when consideration is given to the following detail description thereof. Such description makes reference to the annexed drawings wherein:

FIG. 1 is a planar view showing a stent covered in hydrogel depicted as misformed circles or beads.

FIG. 2 depicts a cross-section of the embodiment of the present invention shown in FIG. 1, situated within in a mammalian vessel, without showing the delivery system.

Note that hydrogel coating is depicted in the foregoing Figures as misformed circles or beads as being representative only, and said circles or beads shown are not drawn to scale

### DETAILED DESCRIPTION OF THE INVENTION

The present disclosure teaches the placement of hydrogel within or coating surfaces of intravascular devices and stents, which are often delivered proximally to target area using a stent allowing for the implementation of a therapeutic endovascular treatment.

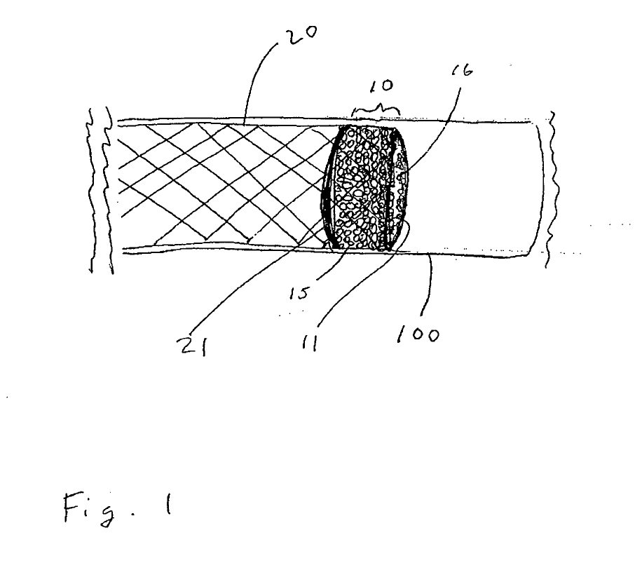

Referring now to FIG. 1, a stent 10 disposed upon a delivery device 20. Stent 10 is shown deployed within vessel wall 100.

Stent 10 includes a distal end 11. Delivery device 20 has a distal end 21.

Stent 10 is coated with hydrogel 15 or 16. Hydrogel 15 or 16 is typically amorphous. It is adhered to all or select surfaces of stent 10 or other intravascular device.

In a preferred embodiment, stent 10 is covered with a one (1) nanometer to one (1) millimeter layer of hydrogel 15 or 16 to prevent thrombosis and tissue reactions. Another embodiment includes impregnating medications into hydrogel 15 or 16 on stent 10. This alternate embodiment may have multiple subgroups including chemotherapy and vasodilator agents, among others. This embodiment could also have multiple applications for treatment of cancer, vasospasm, and other diseases, with varying the medications and the location of the stent.

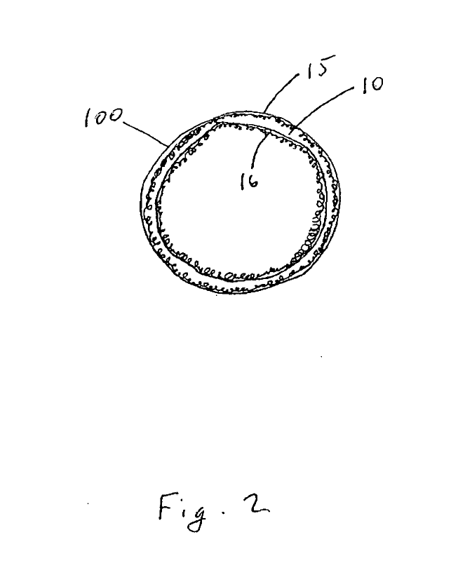

Referring now to FIG. 2, a cross-section of deployed covered stent 10 within vessel wall 100, showing hydrogel 15 or 16 coating on the outer surface 15 and the inner surface 16.

When said coated device 10 is employed in an endovascular treatment, the exposure of the adhered added hydrogel 15 or 16 with the device 10 to the blood and temperature in the body causes it to expand further, decreasing the permeability of device 10 to blood and which decreases the risk of the aneurysm rupturing or clots forming and embolizing.

The present invention uses a device designed to facilitate endovascular treatment by coating hydrogel along delivery device 20 to prevent episodes of distal migration due to addition of hydrogel 15 and 16.

In the preferred embodiment of the current invention a thin coating of hydrogel 15 and 16 is placed on all surfaces, including the surface pressing on the vessel wall to reduce the rate of intimal hyperplasia caused by the vessel reacting to the foreign body. This results in a non-obvious benefit of the use of hydrogel 15 and 16 because vasospasms turn cause sub-optimal outcomes, including in some cases the death of the patent.

The thickness of the hydrogel 15 and 16 coating on the stent 10 would be from the minimum possible thickness of approximately one nanometer or less, up to one centimeter in thickness. However, for most carotid and vertebral artery applications the preferred thickness is one millimeter or less.

In general dosage depends on the specific medication and the intended task. For example, Verapamil 2 mg/hr and Cardene 100 mcg/hr are non-limiting examples of medication doses that are released to the blood for cases of vasospasm. Various possible vasodilators, including and not limited to the ones listed herein may be infused for treating vasospasms; any chemotherapy agent may be employed for treatment of cancer.

In alternate embodiments (not shown), hydrogel 15 and 16 is lined onto nonvascular stents 10, such as biliary and ureter stents, to reduce rates of in-stent stenosis; and may help anchor the stent 10 in place and prevent stent migration.

In another alternate embodiment (not shown), hydrogel 15 and 16 does not fill the interstices between metal areas of stent 10.

In alternate embodiments, hydrogel may be coated on such devices as a delivery mechanism of medications, which can be immediate release or controlled sustained slow release.

Slow, local release of adhered medications is also useful in treating certain cancers.

In some embodiments, a bio-degradeable hydrogel is employed.

In another embodiment, a non-biodegradable hydrbiogel, that will be permanent, may be employed.

In some embodiments said additional coating includes chemotherapy compounds in said thin coating of hydrogel. As examples, said chemotherapy compounds embedded a device may be used in the carotid artery for a brain tumor in that vascular distribution, or in right renal artery for a right kidney tumor, or in right pulmonary artery for a right lung mass. This could allow sustained delivery locally, while minimizing the systemic dose and associated side effects.

Said hydrogel thin coating may be impregnated with pharmaceutical compounds to ameliorate vasospasm. Said compounds may include, but are not limited to nimodipine, Verapamil, Cardene, nitroglycerin, and nitroprusside. Said compounds may be formulated for immediate release or controlled sustained slow release.

By way of non-limiting example, impregnating hydrogel adhered to a stent with Verapamil that is released over two weeks, and placing said stent in carotid artery, may be used to treat intracranial vasospasm. In addition or in the alternative, impregnating hydrogel adhered to a stent with a slow release chemotherapy agent, allowing selective delivery over a time to a single organ, with lower systemic doses, is likely to lead to fewer side-effects. It may allow higher and more effective local doses of medication as well.

To minimize the risk of severe symptomatic vasospasm in aneurysmal subarchnoid hemorrhage (a typical bleed from a ruptured brain aneurysm) or other intracranial vasoconstriction syndrome, the said thin coating of hydrogel might include a vasodilator compound that slowly releases over two to four weeks. Said medication infused hydrogel can be embedded in a stent for placement in the common or internal carotid arteries on one or both sides, and/or the placement in one or both vertebral arteries. Non-limiting examples of vasodilators that can be embedded include nimodipine, Verapamil, Cardene, nitroglycerin, and nitroprusside. They can be implanted therapeutically after vasospasm is identified. In some cases they can be implanted prophylactically, before the onset of vasospasm.

The objective of the present invention is to deliver pharmaceutical compound(s) downstream from the stent 10. The present invention teaches four techniques to achieve this objective in a manner which is superior to the prior art.

First, the hydrogel 15 and 16 which contains the pharmaceutical compounds is located both inside and outside the stent 10 wall. Said positioning allows blood flowing through stent 10 to leach medication from hydrogel 15 or 16. Said blood flow then delivers said pharmaceutical compounds downstream of stent 10. Hydrogel 16 (interior surface) differs from the prior art because the prior art deposits the pharmaceutical compound directly on the outer surface of a (typically) metal stent.

When the prior art devices release the pharmaceutical compounds, they expose metal surfaces either inside the prior-art stent, thus harming the blood, or outside the metal stent, thus harming the vessel tissue in contact with the vessel wall 100. In light of the fact that it is known that exposure of blood to metal causes injury, the present invention in its preferred embodiment uses an intermediate compound such as hydrogel 15 or 16, which covers the stent 10 both inside and out, after the medication has been leached away, thus preventing injury of the metal contact with either the blood or the vessel wall 100. Coating hydrogel 16 inside stent 10 allows superior downstream results as compared to prior-art devices.

The second technique to enhance superior downstream results, when compared to the prior art, is the present invention's asymmetrical distribution of the hydrogel 15 and 16, in some embodiments. By placing relatively more hydrogel 15 and 16 toward the downstream end of stent 10, pharmaceutical compounds are more likely to exit stent 10 and travel further from stent 10 then if the compounds were uniformly distributed on a stent 10, as disclosed by the prior art.

The third technique, in some embodiments, is to shape the inside of stent 10 to produce spiral flow. Said shaping may be achieved by either forming internal spiral ridges on the inside of stent 10, or plating the inside of stent 10 with spiral mounds of hydrogel 15 and 16. Either the spiral ridges coated with hydrogel or spiral mounds of hydrogel will result in changing the course of the blood flow through stent 10. More particularly, such spiral-coated structures or mounds will cause the blood flow through stent 10 to spiral. Such spiraling will encourage turbulent flow. Turbulent flow has a well-known characteristic of clumping particulate matter such as pharmaceutical compounds in the center of the flow. Particulate matter in the center of the flow will go further downstream than particulate matter under laminar-flow conditions.

The fourth technique, in some embodiments, is to make the downstream stent 10 opening smaller than the upstream stent 10 opening. It is well known that constricting a fluid results in turbulent flow. Therefore, for the reasons noted above, pharmaceutical compounds will travel in the center of the flow further downstream than stents with similarly sized openings, as disclosed by the prior art.

The foregoing four techniques, individually or in a combination of one or more of these techniques, may also be used to control the amount and distance the pharmaceutical compounds will be sent downstream in addition to allowing superior downstream range of pharmaceutical-compound delivery as compared to the prior art.

The present invention can alternatively be used by embedding or impregnating pharmaceutical compounds medications in stent 10 for local delivery, short release or sustained release, using permanent non-degradeable hydrogel or biodegradable hydrogel. The following are non-limiting embodiments.

Placing a stent with chemotherapy embedded into carotid artery for a brain tumor in that vascular distribution, or in right renal artery for a right kidney tumor, or in right pulmonary artery for a right lung mass. This could allow sustained delivery locally, while minimizing the systemic dose and associated side effects.

Similarly, to minimize the risk of severe symptomatic vasospasm in aneurysmal subarchnoid hemorrhage (a typical bleed from a ruptured brain aneurysm), a vasodilator that slowly releases over time can be embedded in stent 10 for placement in the common or internal carotid arteries on both sides, with optional additional placement in one or both vertebral arteries. Non-limiting examples of vasodilators that can be embedded include nimodipine, Verapamil, Cardene, nitroglycerin, and nitroprusside.

Although the invention has been described in detail in the foregoing embodiments and methods for the purpose of illustration, it is to be understood that such detail is solely for that purpose, and that variations can be made therein by those skilled in the art without departing from the spirit and scope of the invention, except as it may be described by the following claims.

What is claimed is:

1. A device for ameliorating thrombosis and tissue reactions by deploying a thin coating of hydrogel.

2. The device of claim 1, wherein said hydrogel is deployed upon the outer surface of a stent.

3. The device of claim 1, wherein said hydrogel is deployed upon the inner surface of a stent.

4. The device of claim 1, wherein said hydrogel is deployed upon the interstices in the surface of a stent.

5. The device of claim 1, wherein said hydrogel is impregnated with at least one pharmaceutical compound.

6. The device according to claim 5, for wherein said at least one pharmaceutical compound is released into the blood at a prescribed dose over a prescribed time.

7. The device of claim 1, wherein said device said hydrogel is coated upon any surface exposed to blood or a lumen wall.

8. The device according to claim 1, wherein said hydrogel is coated upon all surfaces of a stent.

9. The device according to claim 1, wherein said hydrogel is deployed upon a stent and expands to occlude the intersteces of said stent.

10. The device according to claim 1, wherein said hydrogel is deployed upon a stent but does not expand sufficiently to occlude the intersteces of said stent.

11. The device of claim 1, wherein said hydrogel is biodegradable.

12. The device of claim 1, wherein said hydrogel is non-biodegradable.

13. The device according to claim 1, wherein said hydrogel is deployed upon a cardiac prosthesis.

14. The device according to claim 1, wherein said hydrogel is deployed upon an orthopedic device.

15. A device comprising a stent for deployment in a body lumen, a coating adhered to said stent, wherein said coating is capable of containing at least one pharmaceutical compound, releasing said at least one pharmaceutical compound into said body lumen for downstream therapy.

16. The device of claim 15, wherein said coating is a hydrogel.

17. The device of claim 15, wherein the device is adapted for intravascular use.

18. The device of claim 15, wherein said device is adapted for use in the genitourinary tract.

19. The device of claim 15, wherein said device is adapted for use in the biliary tract.

20. The device of claim 15, wherein said device is adapted for use in a gastrointestinal tract.

21. The method of treating vasospasm via implanting at least one stent that releases at least one vasodilator pharmaceutical into a vascular territory.

22. The method of prophylactively ameliorating post-aneurysmal vasospasm via implanting at least one stent that releases at least one vasodilator pharmaceutical into a vascular territory.

23. The method of delivering at least one chemotherapy via implanted stents impregnated with at least one chemotherapy agent.
